(19) United States
(12) Patent Application Publication
(10) Pub. No.: US 2026/0249097 A1
(43) Pub. Date: Aug. 27, 2026

(54) METHODS AND DEVICES FOR WOUND HEALING

(71) Applicant: Gary Goodman, Longmeadow, MA (US)

(72) Inventor: Gary Goodman, Longmeadow, MA (US)

(21) Appl. No.: 19/459,380

(22) Filed: Jan. 26, 2026

Related U.S. Application Data
(60) Provisional application No. 63/833,942, filed on Jan. 29, 2025.

(51) Int. Cl. A61N 5/06; A61B 18/20

(52) U.S. Cl. CPC A61N 5/0616; A61N 5/0624; A61B 18/203; A61N 2005/0659; A61N 2005/0663

(57) ABSTRACT

In one aspect, the invention provides a method for wound healing. The method comprises exposing the wound to a first electromagnetic radiation, and then exposing the wound to a second electromagnetic radiation. The order of exposure may be reversed, or the exposure can be done simultaneously. The power of the first electromagnetic radiation is such that it achieves penetration to the deeper layers of the wound, all the way to the wound bed. Useful power range in the invention is about 1 Watts to about 1000 Watts, and an energy density for a given treatment session ranges from about 1 mJ/cm² to about 1000 J/cm². The invention also provides a device for wound healing that comprises a first and a second electromagnetic radiation source. The method and device of the invention may involve use of a third and/or more electromagnetic radiation also.

### TECHNICAL FIELD OF THE INVENTION

The invention relates generally to devices and methods for wound healing and more specifically to exposing wounds to electromagnetic radiation of two or more different wavelengths to enable healing.

### BACKGROUND

Wound results from a number of different causes and results in an acute injury to the epidermis of the skin, and depending on the nature of the wound, may even extend to the dermis and/or the hypodermis layers. Wounds need to be healed as quickly as possible so that it does not lead to further complications. If left unchecked, it can become infected due to contamination.

Wound healing may be hampered for certain individuals due to various reasons. For example, about 6.5 million Americans are estimated to be diabetic and have chronic or non-healing wounds. When left unchecked, unhealed wounds may result in amputations or may be the cause of morbidity.

Use of low level laser therapy for healing both late stage as well as early stage wounds is known. However, the techniques known currently in the art are specific one or the other type of wound. The low level laser therapy requires several sessions of laser exposure, and is extremely contingent upon patient's compliance between sessions. This may not always be possible for a number of different reasons, and consequently, wound healing is delayed considerably or does not heal at all.

There remains a dire need to develop a non-invasive technique to heal wounds rapidly with very little or no difficulty to the patient.

### BRIEF DESCRIPTION OF THE INVENTION

In one aspect, the invention provides a method for wound healing. The method comprises exposing the wound to a first electromagnetic radiation having a first wavelength in the infrared region of the electromagnetic spectrum. The first wavelength may be ranging from about 800 nanometers to about 1300 nanometers. The method then comprises exposing the wound to a second electromagnetic radiation having a second wavelength in the blue region of the electromagnetic spectrum. The second wavelength may be ranging from about 400 nanometers to about 500 nanometers. The second wavelength is preferably 450 nanometers. In another embodiment, the method of the invention may involve exposure of the wound to the second electromagnetic radiation followed by the first electromagnetic radiation. In yet another embodiment, the method of the invention may involve simultaneous exposure of the wound to the first and second electromagnetic radiations. The method may further comprise exposing the wound to a third electromagnetic radiation having a third wavelength in the red region of the electromagnetic spectrum. The third wavelength may be ranging from about 600 nanometers to about 700 nanometers. In some embodiments, a fourth electromagnetic radiation, and further additional electromagnetic radiations are used for wound treatment.

The power of the first electromagnetic radiation is such that it enables penetration of the radiation all the way to the dermis layer, or even the hypodermis layer. The power may range from about 1 milliWatts to about 1000 Watts. The time of exposure may vary depending on the power, the size of the wound, stage of the wound, size of the radiation spot, and the like, and combinations thereof. The total energy density of all the electromagnetic radiation ranges from about 1 mJ/cm² to about 1000 J/cm². In a preferred embodiment, the total energy density of all the electromagnetic radiation ranges from about 10 mJ/cm²—to about 25 mJ/cm².

In another aspect, the invention provides a device for wound healing. The device comprises a first electromagnetic radiation source and a second electromagnetic radiation source, wherein the first and second electromagnetic radiation are as described herein. The device further comprises a power source to provide power. The device may further comprise a third and/or more number of electromagnetic radiation source to provide a third and/or more electromagnetic radiation as described herein.

In yet another aspect, the invention provides a system for wound healing, wherein the system comprises the device for wound healing as described herein. The system comprises a memory unit, a controller unit, an input unit and an output unit. The memory unit comprises a memory chip that comprises instructions configured to run a program that the controller unit would execute to control the device of the invention.

In a further aspect, the invention provides a method for controlling the device for wound healing as described herein.

### BRIEF DESCRIPTION OF DRAWINGS

FIG. 1 shows a photograph of an exemplary test setup within a biosafety cabinet in accordance with the method of the invention.

FIG. 2 shows a photograph of agar plates (including positive control) comprising P. aeruginosa after exposure to laser treatment for 24 hours.

FIG. 3 shows a photograph of agar plates (including positive control) comprising E. coli after exposure to laser treatment for 24 hours.

FIG. 4 shows a photograph of agar plates (including positive control) comprising S. aureus after exposure to laser treatment for 24 hours.

FIG. 5 shows a photograph of each plate post subculture and incubation.

FIG. 6 shows a photograph of agar plates and subculture plates post incubation comprising P. aeruginosa after exposure to laser treatment for 24 hours.

FIG. 7. shows a photograph of agar plates and subculture plates post incubation comprising E. coli after exposure to laser treatment for 24 hours.

FIG. 8 shows a photograph of agar plates and subculture plates post incubation comprising S. aureus after exposure to laser treatment for 24 hours.

### DETAILED DESCRIPTION

The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the term “or” is generally employed in its sense including “and/or” unless the content clearly dictates otherwise.

As used herein, the term “electromagnetic radiation source” refers to an element of the electromagnetic radiation therapy apparatus that is configured to provide an optical output (e.g., to transmit light from a light therapy apparatus to a target tissue, such as an ocular tissue, of a patient). These may include any type of light sources, which comprises, for example, but not limited to, light emitting diodes (LEDs), laser diodes, intense pulsed light, and the like, and combinations thereof. The emitted electromagnetic radiation is characterized by a number of parameters, such as wavelength, intensity, amplitude, and the like. In some embodiments, the electromagnetic radiation has a specific wavelength, while in other embodiments, the electromagnetic radiation has a band of wavelengths.

As noted herein, in one aspect the invention provides a method of treating wounds. The method comprises exposing the wound to a first electromagnetic radiation having a first wavelength in the infrared region of the electromagnetic spectrum. In particular embodiments, the first electromagnetic radiation has a wavelength ranging from about 800 nanometers to about 1300 nanometers. The method then comprises exposing the wound to a second electromagnetic radiation having a second wavelength in the blue wavelength range. In particular embodiments, the second electromagnetic radiation has a wavelength ranging from about 400 nanometers to about 500 nanometers. In some embodiments, the first and/or second electromagnetic radiation has a single specific wavelength, while in other embodiments, first and/or second electromagnetic radiation has a broad range of wavelengths that may be known as a band of wavelengths.

In some specific embodiments, the first electromagnetic radiation has a wavelength ranging from about 950 to about 1064 nanometers, and in very specific embodiments, the first electromagnetic radiation has a wavelength of 980 nanometers, and the second electromagnetic radiation has a wavelength of 450 nanometers. The second electromagnetic radiation may be configured to have a power in a range that allows for penetration into the depth of the wound or may be configured to act on the surface only.

Further, the method of the invention may involve exposure of the wound to a third electromagnetic radiation having a third electromagnetic wavelength in the red region of the electromagnetic spectrum, which may be ranging from about 600 nanometers to about 700 nanometers. The third electromagnetic radiation may also have a specific wavelength or may have a band of wavelengths. The method may comprise a fourth and further electromagnetic radiations having suitable wavelength used to treat the wound. In some instances, the same electromagnetic radiations may be used to cycle through a particular treatment session in a certain order. In specific embodiments, the third electromagnetic radiation has a wavelength of about 650 nanometers.

All the electromagnetic radiations may be made available conveniently as lasers having the appropriate wavelengths and the useful power and/or intensity. The laser may be made available as continuous wave (CW) or pulsed lasers, the choice of which will become apparent to one skilled in the art. In some embodiments, the power of the lasers used in the first and second electromagnetic radiation independently ranges from about 1 Watt to about 200 Watts.

The use of low intensity lasers at a wavelength of 980 nanometers for wound healing is well known. The penetration of the electromagnetic radiation having wavelength in the infrared region (like 980 nm) is much better and therefore the activation of heat shock proteins and new vascularization is better. On the other hand, the more shallow penetration of the blue wavelength band (400-480 nm) leads to a direct damage of bacteria that are potentially in the superficial part of the wound and contribute to disinfection which can be difficult through other means, such as a spray that is designed to act chemically to eliminate bacteria and viruses. Hessling et al (M. Hessling, B. Spellerberg, K. Hoenes, Photoinactivation of bacteria by endogenous photosensitizers and exposure to visible light of different wavelengths—a review on existing data, FEMS Microbiology Letters, Volume 364, Issue 2, January 2017, fnw270, https://doi.org/10.1093/femsle/fnw270) have observed that the use of electromagnetic radiation having wavelengths 405 nm and 470 nm have the effect of inactivating bacteria. In a different study (Prado, T. P.; Zanchetta, F. C.; Barbieri, B.; Aparecido, C.; Melo Lima, M. H.; Araujo, E. P. Photobiomodulation with Blue Light on Wound Healing: A Scoping Review. Life 2023, 13, 575. https://doi.org/10.3390/life13020575), Prado et al have shown that electromagnetic radiation having wavelength ranging from about 405 nm to about 470 nm has photobiomodulating effect on cells, i.e. depending on certain parameters, it can proliferate or reduce cell growth. Hence, it can be postulated that electromagnetic radiation having wavelengths ranging from about 400 nm to about 500 nm can be used effectively for disinfecting wounds, cleaning wound sites, and even heal wounds by proliferation of cell growth.

U.S. Pat. No. 7,177,695 B2 discloses the use of electromagnetic radiation having wavelength of 980 nanometers and a power ranging from about 1 W to about 20 W for the treatment of early stage wounds. This is applicable specifically for treatment of stage 1 wounds, or in some instances, stage 2 wounds. It is well known that early stage wounds are associated with little or no breach of the skin tissue. Consequently, the electromagnetic radiation need not penetrate the skin layers, and instead be functional only on the surface layers.

However, in the case of later stage wounds, where there is substantial damage to the deeper layers of the dermis and possibly even hypodermis, the methods and devices known and described in the prior art are inadequate. It is understood that electromagnetic radiation at a low power is not capable of penetrating the skin layers. However, a higher power laser with the same wavelength can penetrate the skin layers and have an effect on the wound bed.

Without being bound to any theory, it is speculated that the electromagnetic radiation having a wavelength 980 nanometers also acts as a disinfecting agent. This has been noted earlier in the '775 patent also. In the present invention, the disinfecting effect of the 980 nanometers laser is combined with the depth penetrating ability of the appropriately powered laser to give rise to a surprising ability of disinfecting the wound bed as well as the surface of a wound. For wound healing it is important that 980 nanometers radiation be present in low or high power as long as it sufficient to instantly kill bacteria. Varying intensities, and densities of the radiation affects the penetration from shallow to deep tissue killing microorganisms such as bacteria. Thus, bacteria present even in deeper parts of the wound tissue will be eliminated through the use of the electromagnetic radiation of the invention. Bacteria in the deeper tissue are often associated with later stage wounds. A clean wound bed allows for revascularization, and associated regrowth of wound tissue. The method of the invention allows for synergistic combination of various wavelengths of electromagnetic radiation mentioned herein which would otherwise not be possible by the use of single wavelengths.

Conventionally, a wound is debrided with a debridement tool (typically a steel blade) to remove necrotic (dead) tissue and prepare the wound bed. This is a manual procedure that requires tool handling with extreme care and caution. The procedure is associated with trauma and hence prone to cause further damage to the wound, which may slow down the healing process.

In this invention, the electromagnetic radiation is also used to debride the wound. This will result in debridement of bacteria and fungus and prepare the wound bed. The use of electromagnetic radiation negates the problems associated with the use of any debridement tool. In some cases, the use of the debridement tool maybe necessary only in the first instance, following which the debridement can be achieved by the use of first electromagnetic radiation. Further, repeated formation of necrotic tissue requiring constant removal would suggest a sub-optimal wound healing. In this situation, debridement using electromagnetic radiation would be the best course of action, sometimes only course of action, for enhancing wound healing. Hence, wound treatment would include an electromagnetic radiation as a standard debridement tool. The first electromagnetic radiation can thus be used for debriding and/or disinfecting the wound.

Thus, one skilled in the art will understand that the intensity or power of the first electromagnetic radiation is chosen such that the radiation is capable of penetrating the wound right down to the wound bed. The time of exposure of the wound to the first electromagnetic radiation is dependent on various factors, such as, but not limited to, power of the electromagnetic radiation, wavelength, size of the wound, early stage or late stage of the wound, other wound characteristics, and so on, and combinations thereof. Based on some of the known parameters, one skilled in the art can build a graph or a suitable look-up table between power and time of exposure for a given stage of wound, for example. On the basis of this graph or look-up table, for a given power of the first electromagnetic radiation, a time of exposure can be determined. The time of exposure and power of the electromagnetic radiation are also correlated with the wavelength of the radiation, as well as depth of penetration of the radiation. Thus, a calibration curve, or the look-up table, correlating the various parameters related to the electromagnetic radiation may be developed, and accordingly, for a given value of the parameters, the missing parameter can be arrived at from the calibration curve or the look-up table. In some exemplary embodiments, the time of exposure ranges from about 1 millisecond to about 6 hours. Such a decision making capability can also be programmed into a chip that can be incorporated into a suitable device of the invention, which would enable automated choices for a particular treatment.

As already described herein, the time of exposure can also be arrived at without undue experimentation given other parameters such as the intensity, power, wavelength, size of wound, stage of wound, diameter of the laser spot, and the like, and combinations thereof. Other such parameters to determine the time of exposure, depth of exposure, etc. will become obvious to one skilled in the art, and is contemplated to be within the scope of the invention.

The total energy density of all the electromagnetic radiation for a given treatment session ranges from about 1 mJ/cm² to about 1000 J/cm². This can be arrived at using methods as described herein, based on a calibration curve derived for the treatment session. The total energy density is a cumulative energy density of all the electromagnetic radiation used for the exposure during a specific treatment session.

The method of the invention, in another aspect, comprises exposing the wound to the second electromagnetic radiation followed by the first electromagnetic radiation. In yet another aspect, the method comprises exposing the wound to the third electromagnetic radiation followed by the first electromagnetic radiation and then the second electromagnetic radiation. In yet another aspect, the method of the invention comprises exposing the wound to the first electromagnetic radiation followed by the third electromagnetic radiation and then the second electromagnetic radiation. In a further aspect, the method comprises exposing the wound to the third electromagnetic radiation followed by the second electromagnetic radiation and then the first electromagnetic radiation.

In another aspect, the invention provides a device for wound healing. The device comprises a first electromagnetic radiation source to provide the first electromagnetic radiation and a second electromagnetic radiation source to provide the second electromagnetic radiation, wherein the first and second electromagnetic radiation are as described herein. The device further comprises a power source to provide power. The device comprises a third electromagnetic radiation source to provide the third electromagnetic radiation as described herein. In some embodiments, the device may include fourth and/or more electromagnetic radiation of suitable wavelengths that is used to treat wounds. The device is configured to provide the first, second, the third, and/or the fourth and further electromagnetic radiations in any particular order, along with the optimal power requirements, and the total energy density, as desired for a specific treatment protocol as described herein. The device of the invention provides for the use of a synergistic combination of wavelengths of electromagnetic radiation for effective wound healing that is otherwise not feasible.

In yet another aspect, the invention provides a system for wound healing, wherein the system comprises the device for wound healing as described herein. The system comprises a memory unit, a controller unit, an input unit and an output unit. The memory unit comprises a memory chip that comprises instructions configured to run a program that the controller unit would execute to control the device of the invention. The input unit is configured to take inputs related to treatment of the wound. Inputs may include parameters such as, but not limited to, patient details such as, for example, name, age, prior conditions, past treatment details, medical history, etc.; electromagnetic radiation power; time of exposure; spot diameter; spot shape; area of exposure; pattern of exposure; and the like. The system may also be configured to take images of the wound before, during and after the exposure. The images may be displayed on the output unit. Further, infrared images may also obtained before, during, and after treatment sessions. Based on the infrared images obtained, the areas requiring greater attention may be gleaned. Based on the infrared images, regions requiring higher intensity and lower intensity, regions requiring longer exposure versus shorter exposure, selected specific wavelengths for specific regions, and such specific variations of treatment within the region of the wound can also be obtained. Further, selected specific electromagnetic radiation may be applied within a region based on thermal imaging signaling. Thus, a specific treatment plan can be arrived at based on digital images as well as infrared images. Patient details, treatment session details, associated images may be stored on the memory unit, which may be displayed on the output unit when called upon using the appropriate instructions. The access to the details may be restricted according to the access level of the user, such levels may include, for example, administrator, owner, manager, user, etc.

Thus, the invention also provides a method for controlling the device of the invention using the system of the invention as described herein.

### EXAMPLES

### Laser Exposure on Bacterial Cultures

### Objective

The objective of this bench study is to evaluate whether exposure to a 980 nm diode laser and a 450 nm blue laser has an observable effect on bacterial growth when applied to agar plates.

### Organisms

The following bacterial cultures were used:
1) Escherichia coli (ATCC 10798)
2) Pseudomonas aeruginosa (ATCC 10145)
3) Staphylococcus aureus (ATCC 12598)

### Materials

Agar plates inoculated with the above organisms
Laser sources (980 nm and 450 nm wavelengths)
Standard laboratory protective equipment (lab coat, gloves, eye protection)
Biosafety Cabinet
Incubator

### Experimental Design

Each of the three bacterial strains was cultured on its recommended ATCC reference medium. Cultures were plate-spotted and incubated for 24 hours to obtain visible growth. For each strain, three test cultures and one positive control were prepared.

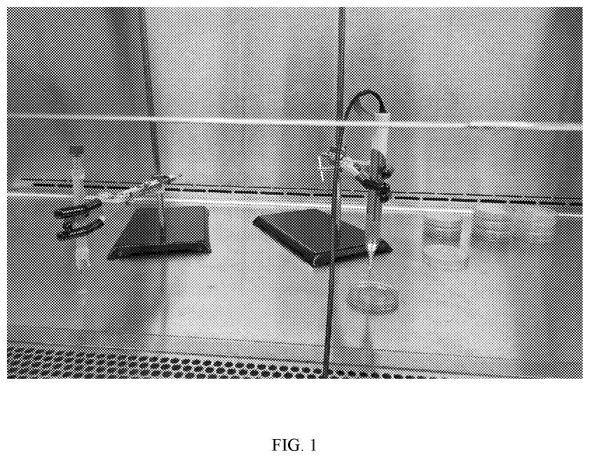

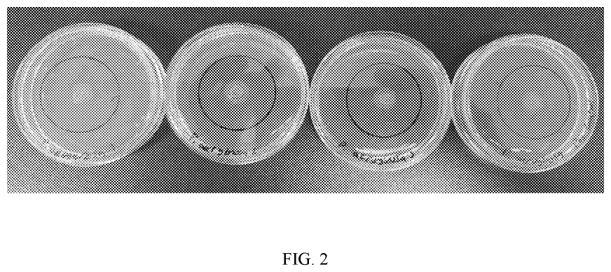

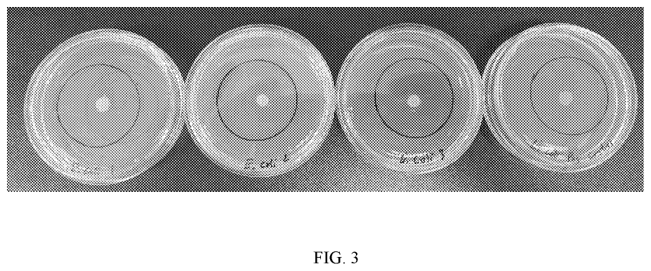

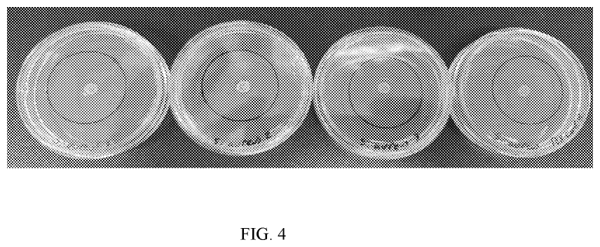

Bacterial growth was then sequentially exposed to a 980 nm laser followed by a 450 nm laser, according to the parameters outlined in Table 1. All procedures were conducted inside a biosafety cabinet to maintain aseptic conditions (see FIG. 1). Positive controls were not exposed to laser treatment, and negative control plates containing no bacteria were also included. FIGS. 2-4 show photographs of the three test plates for each strain following laser exposure, alongside the untreated positive control for comparison.

TABLE 1
Laser Parameters

| Wavelength | Exposure Surface | Exposure Parameters |
|---|---|---|
| 980 nm | 1 cm diameter circle | 5 W, 46 secs, 293 J/cm2 |
| 450 nm | 1 cm diameter circle | 2 W, 106 sec, 270 J/cm2 |

Following laser exposure, each test culture was subcultured onto fresh plates using a sterile loop. The same procedure was applied to both positive and negative controls. The plates were then incubated for 7 days and monitored for bacterial growth.

### Results

No bacterial growth was observed on any plates that had been subcultured from the bacterium exposed to the 980 nm and 450 nm laser treatments across all three strains tested (E. coli, P. aeruginosa, and S. aureus). In contrast, growth was observed on the positive control plates for each strain, confirming culture viability. As expected, no growth was detected on the negative control plates. These findings indicate that laser exposure under the tested conditions inhibited bacterial growth, while the control groups performed as anticipated.

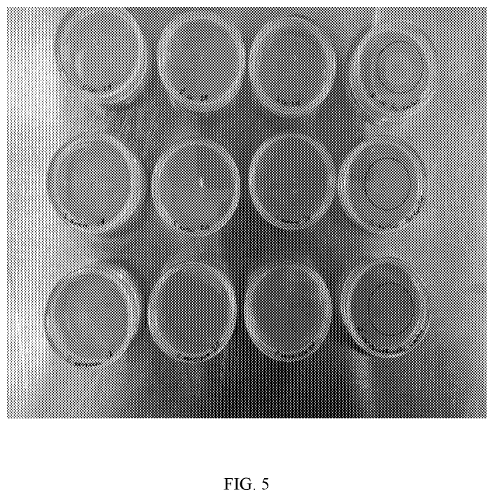

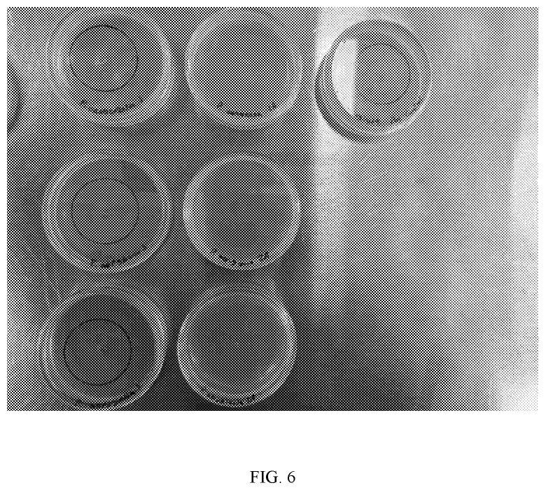

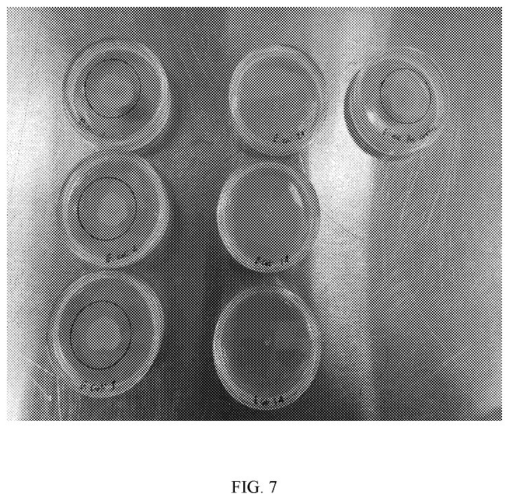

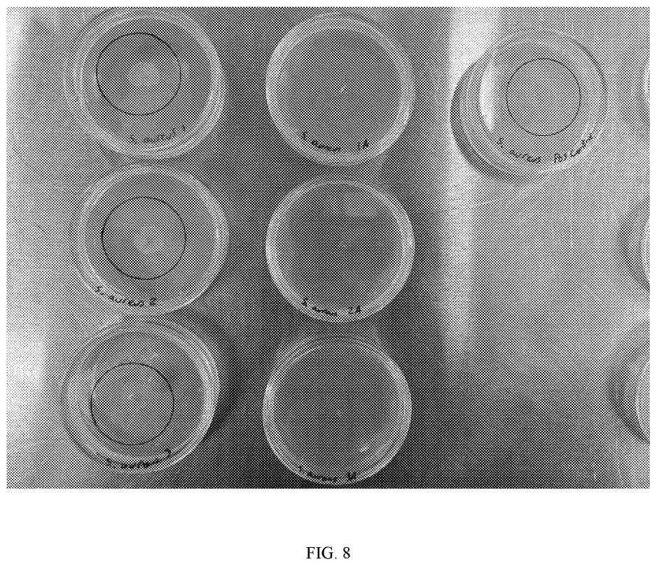

FIGS. 5-8 show photographs of each of the plates after incubation. Plates 1, 2, and 3 for each strain were the ones exposed to the laser treatment while plates 1A, 2A, and 3A were the ones the bacterium exposed to the laser was subcultured onto. The positive control in the figures below are the subcultured plates from the non-exposed 24 hour bacterium culture.

### CONCLUSION

Exposure of E. coli, P. aeruginosa, and S. aureus to sequential 980 nm and 450 nm laser treatment resulted in complete inhibition of bacterial growth under the conditions tested. Positive and negative controls behaved as expected, supporting the validity of the findings. These results suggest that laser irradiation at the tested wavelengths effectively killed bacteria directly exposed to the laser and prevented subsequent growth when the exposed bacterium was transfer to new growth medium. The results further show that the combinations of wavelengths of laser radiation rendered the killing of bacteria possible as opposed to the use of a single wavelength of radiation.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method for wound healing, the method comprising:
exposing the wound to a first electromagnetic radiation; and
exposing the wound to a second electromagnetic radiation.

2. The method of claim 1, wherein the first electromagnetic radiation has a wavelength ranging from about 800 nanometers to about 1300 nanometers.

3. The method of claim 1, wherein the second electromagnetic radiation has a wavelength ranging from about 400 nanometers to about 500 nanometers.

4. The method of claim 1 wherein the second electromagnetic radiation has a wavelength of about 450 nanometers.

5. The method of claim 1, wherein the exposure to the second electromagnetic radiation is done after the exposure to the first electromagnetic radiation.

6. The method of claim 1, wherein the exposure to the first electromagnetic radiation is done after the exposure to the second electromagnetic radiation.

7. The method of claim 1, wherein the exposure to the first and second electromagnetic radiation is done simultaneously.

8. The method of claim 1, further comprising exposing the wound to a third electromagnetic radiation, wherein the third electromagnetic radiation has a wavelength ranging from about 600 nanometers to about 700 nanometers.

9. The method of claim 8, wherein the third electromagnetic radiation has a wavelength of about 640 nanometers.

10. The method of claim 1, wherein the first electromagnetic radiation has a power ranging from about 1 Watt to about 1000 Watts.

11. The method of claim 1, wherein a energy density for a given treatment session ranges from about 1 mJ/cm² to about 1000 J/cm².

12. The method of claim 1 further comprising a debridement step, wherein the debridement step is effected using an electromagnetic radiation.

13. A device for wound healing, the device comprising:
a first electromagnetic radiation source to provide a first electromagnetic radiation; and
a second electromagnetic radiation source to provide a second electromagnetic radiation.

14. The device of claim 13, wherein the first electromagnetic radiation has a first wavelength ranging from about 800 nanometers to about 1300 nanometers.

15. The device of claim 13, wherein the second electromagnetic radiation has a second wavelength ranging from about 400 nanometers to about 500 nanometers.

16. The device of claim 13, further comprising a third electromagnetic radiation source to provide a third electromagnetic radiation having a wavelength ranging from about 600 nanometers to about 700 nanometers.

17. The device of claim 13, wherein the first electromagnetic radiation has a power ranging from about 1 milliWatts to about 1000 Watts.

18. The device of claim 16 wherein the device is capable of generating the first, the second and the third electromagnetic radiation simultaneously, or in any sequence.

19. A system comprising the device of claim 13.

20. A method for wound healing, the method comprising:
exposing the wound to a second electromagnetic radiation; and
exposing the wound to a first electromagnetic radiation.

21. A method for wound healing, the method comprising:
exposing the wound to a first electromagnetic radiation;
exposing the wound to a third electromagnetic radiation; and
exposing the wound to a second electromagnetic radiation.

22. A method for wound healing, the method comprising:
exposing the wound to a second electromagnetic radiation;
exposing the wound to a first electromagnetic radiation; and
exposing the wound to a third electromagnetic radiation.

23. A method for wound healing, the method comprising:
exposing the wound to a second electromagnetic radiation;
exposing the wound to a third electromagnetic radiation; and
exposing the wound to a first electromagnetic radiation.

24. A method for wound healing, the method comprising:
exposing the wound to a third electromagnetic radiation;
exposing the wound to a first electromagnetic radiation; and
exposing the wound to a second electromagnetic radiation.

25. A method for wound healing, the method comprising:
exposing the wound to a third electromagnetic radiation;
exposing the wound to a second electromagnetic radiation; and
exposing the wound to a first electromagnetic radiation.

26. A method for wound healing, the method comprising:
exposing to a first electromagnetic radiation, a second electromagnetic radiation, and a third electromagnetic radiation simultaneously.
